(19) United States
(12) Plant Patent Application Publication
(10) Pub. No.: US 2003/0088895 P1
(43) Pub. Date: May 8, 2003

(54) Floribunda rose plant named 'Olijdum'

(76) Inventor: Huibert W. Olij, De Kwakel (NL)

Correspondence Address: BURNS, DOANE, SWECKER & MATHIS, L.L.P., P.O. Box 1404, Alexandria, VA, 22313-1404, US

(21) Appl. No.: 10/306,408

(22) Filed: Nov. 29, 2002

Related U.S. Application Data
(63) Continuation of application No. 09/293,176, filed on Apr. 16, 1999, now abandoned.

(51) Int. Cl. A01H 5/00

(52) U.S. Cl. PLT/151

(57) ABSTRACT

A new and distinct variety of rose plant of the Spray Class is provided which abundantly forms attractive dark red double blossoms in the form of a well-balanced spray. The buds are globular in configuration and the bud heights are substantially uniform within the spray. An erect growth habit is exhibited. The foliage is light green and contrasts nicely with the dark red blossoms. The plant is well suited for the production of cut floral sprays under greenhouse growing conditions.

### SUMMARY OF THE INVENTION

The new variety of Floribunda rose plant was created by artificial pollination wherein two parents were crossed which previously had been studied in the hope that they would contribute the desired characteristics. The female parent (i.e., the seed parent) of the new variety was the ‘Red Ace’ variety (U.S. Plant Pat. No. 4,840). The male parent (i.e., the pollen parent) of the new variety was the ‘Idole’ variety (non-patented in the United States). The parentage of the new variety can be summarized as follows:

‘Red Ace’×‘Idole’.

The seeds resulting from the above pollination were sown and small plants were obtained which were physically and biologically different from each other. Selective study resulted in the identification of a single plant of the new variety.

It was found that the new variety of Floribunda rose plant of the present invention possesses the following combination of characteristics:

(a) forms in abundance attractive globular buds in the form of a very uniform spray,

(b) forms in abundance attractive dark red double blossoms,

(c) exhibits an erect growth habit, and

(d) forms attractive semi-glossy light green foliage.

Also, the spray inflorescence is well balanced, and its dark red blossom coloration contrasts nicely with the light green foliage. The bud height tends to be substantially uniform within a spray.

The new variety of the present invention can be readily distinguished from its parental varieties. More specifically, ‘Red Ace’ is a Miniature rose that forms smaller delicate flowers, and ‘Idole’ is a Hybrid Tea rose that displays orange-red flowers.

The new variety well meets the needs of the horticultural industry. It is particularly well-suited for use in the production of cut floral sprays under greenhouse growing conditions.

The new variety has been found to undergo asexual propagation in France by a number of routes, including budding, grafting, and cuttage. Asexual propagation by the above-mentioned methods as performed in France has shown that the characteristics of the new variety are strictly transmissible from one generation to another.

The new variety has been named the ‘Olijdum’ variety.

### BRIEF DESCRIPTION OF THE PHOTOGRAPH

The accompanying photograph shows as nearly true as it is reasonably possible to make the same, in a color illustration of this character, typical specimens of the plant parts of the new variety. The rose plants of the new variety were two years of age were observed during April while growing on Rosa indica understock in greenhouses at LeCannet des Maures, Var, France.

FIG. 1—illustrates a specimen of a young shoot;

FIG. 2—illustrates a specimen of a floral bud before the opening of the sepals;

FIG. 3—illustrates a specimen of a floral bud at the opening of the sepals;

FIG. 4—illustrates a specimen of a floral bud at the opening of the petals;

FIG. 5—illustrates a specimen of a flower during the course of opening;

FIG. 6—illustrates a specimen of an open flower—plan view—obverse;

FIG. 7—illustrates a specimen of an open flower—plan view—reverse;

FIG. 8—illustrates a specimen of a fully open flower—plan view—obverse;

FIG. 9—illustrates a specimen of a fully open flower—plan view—reverse;

FIG. 10—illustrates a specimen of a floral receptacle showing the arrangement of the stamens and pistils;

FIG. 11—illustrates a specimen of a floral receptacle showing the arrangement of the pistils (stamens removed);

FIG. 12—illustrates a specimen of a flowering stem;

FIG. 13—illustrates a specimen of a main branch;

FIG. 14—illustrates specimens of two leaves with three leaflets—plan view—upper surface (top) and under surface (bottom);

FIG. 15—illustrates a specimen of a leaf with five leaflets—plan view—under surface; and

FIG. 16—illustrates specimens of two leaves with seven leaflets—plan view—upper surface (left) and under surface (right).

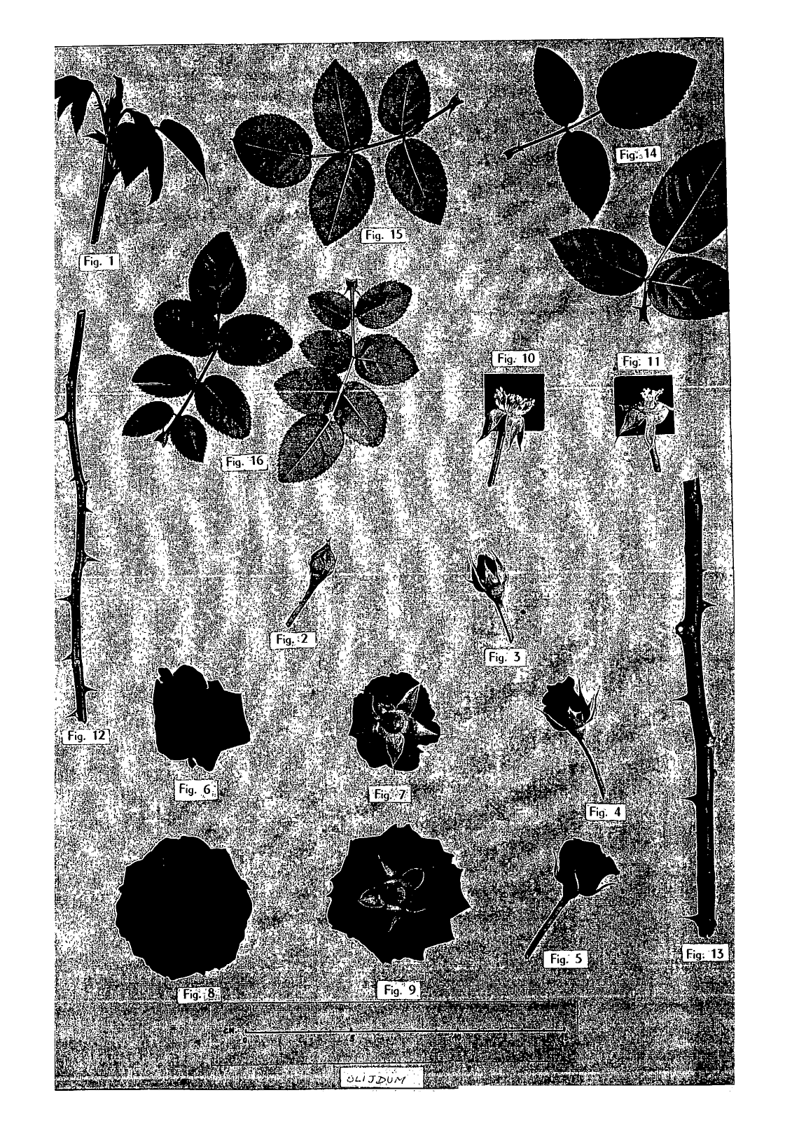

### DETAILED DESCRIPTION

The chart used in the identification of the colors is that of The Royal Horticultural Society (R.H.S. Colour Chart). When more common color terms are utilized they are to be accorded customary dictionary significance. The description is based on two year-old specimens of the new variety which were observed during April while budded on Rosa indica understock and growing in greenhouses at LeCannet des Maures, Var, France. Dimensions in centimeters are provided at the bottom of the photograph.

Class: Floribunda.

Plant:

Height.—a plant pruned to a height of 85 cm. commonly produces floral stems having a length of approximately 40 to 60 cm.

Habit.—erect.

Branches:

Color.—young stems: near Green Group 137A and somewhat suffused with reddish coloration. adult wood: near Green Group 137A.

Thorns.—size: small (as illustrated). quantity: numerous (as illustrated). configuration: substantially straight on the upper surface, and slightly concave on the under surface with a narrow base on the under surface. color: near Greyed-Orange Group 165B on adult wood.

Leaves:

Petioles.—rather glandular on the upper surface, and with a few prickles on the under surface. The coloration is near Green Group 137B on the upper side and near Yellow-Green Group 147B on the under side.

Stipules.—adnate, pectinate, and rather broad.

Leaflets.—number: 3, 5 (most often), and 7. shape: elliptic. texture: rather consistent. serration: regular. general appearance: very dense, light green, and semi-glossy. color (young foliage): upper surface: Green Group 137B and suffused with reddish brown. under surface: Green Group 137B and suffused with reddish brown. color (adult foliage): upper surface: Green Group 137B. under surface: Greyed-Green Group 191A.

Inflorescence:

Number of flowers.—commonly approximately 6 to 15 per stem. The resulting spray is very uniform in the sense that the buds are located close to each other at more or less the same level.

Peduncle.—rigid, near Greyed-Green Groups 191A in coloration, and approximately 5 to 8 cm. in length on average.

Pedicel.—rigid, near Greyed-Green Group 191A in coloration, and approximately 7.5 cm. in length on average.

Sepals.—tomentose and near Greyed-Green Group 191A in coloration with very few extensions.

Buds.—shape: globular. length: approximately 1.5 to 2 cm. on average. size: medium for the Class. color upon opening: upper surface: near Greyed-Purple Group 187A. under surface: near Greyed-Purple Group 187A.

Flower.—form: with somewhat parallel sides. diameter: approximately 6.5 cm. on average. color (when opening begins): upper surface: near Red Group 46A suffused with Red-Purple Group 59A. under surface: near Red Group 46A suffused with Red-Purple Group 60A. color (when blooming): upper surface: near Red Group 46A suffused with Red-Purple Group 59A. under surface: between Red Group 46A and Red Group 53A. color (at end of opening): upper surface: Red Group 46A suffused with Red-Purple Group 59A and with a velvet aspect. under surface: Red Group 53A. fragrance: none. petal number: approximately 35 on average. petal configuration: with undulated margins and a reflexed tip on the outermost petals. petal drop: good, the petals commonly detach cleanly. anthers: yellow-ochre in coloration. stamen number: approximately 80 on average. filaments: yellow in coloration. pistil number: approximately 60 on average. stigmas: yellow in coloration. styles: straw-colored. receptacle: near Greyed-Green Group 191A in coloration, smooth, and in longitudinal section in the shape of a pitcher. lasting quality: approximately 10 to 12 days on the plant, and approximately 10 to 12 days when cut and placed in a vase.

Development:

Vegetation.—strong.

Blooming.—abundant.

Aptitude to bear fruit.—none.

Resistance to diseases.—very good with respect to Botrytis and Mildew.

I claim:

1. A new and distinct variety of spray rose plant characterized by the following combination of characteristics:
(a) forms in abundance attractive globular buds in the form of a very uniform spray,
(b) forms in abundance attractive dark red double blossoms,
(c) exhibits an erect growth habit, and
(d) forms attractive semi-glossy light green foliage;
substantially as herein shown and described, and the parts thereof.
